(19) United States
(12) Patent Application Publication
(10) Pub. No.: US 2014/0113251 A1
(43) Pub. Date: Apr. 24, 2014

(54) COMPOSITE CROWN/COMPOSITE BRIDGE AND METHOD FOR PRODUCTION THEREOF

(71) Applicants: Josef Schweiger, Bergen (DE); Peter Neumeier, Reit im Winkl (DE)

(72) Inventors: Josef Schweiger, Bergen (DE); Peter Neumeier, Reit im Winkl (DE)

(21) Appl. No.: 14/122,993

(22) PCT Filed: May 7, 2012
(86) PCT No.: PCT/EP2012/001951

§ 371 Date: Jan. 7, 2014

(30) Foreign Application Priority Data
Jun. 1, 2011 (DE) 10 2011 103 027.5

(51) Int. Cl. A61C 5/10; A61K 6/04; A61C 13/265; A61C 13/00; A61C 13/15; A61C 13/08; A61C 13/271

(52) U.S. Cl. CPC A61C 5/10; A61C 13/082; A61K 6/04; A61C 13/26; A61C 13/0004; A61C 19/003; A61C 13/2656
USPC 433/199.1; 433/202.1; 433/203.1; 433/206; 433/218

(57) ABSTRACT

The invention relates to a new kind of tooth replacement crown and tooth replacement bridge, of which the components are a framework and a veneer, which are reversibly connected to each other by a macro-retentive bonding system, and a method for producing the components, in which method these components are preferably produced by means of computer-aided design and computer-aided manufacturing.

### CROSS REFERENCE TO RELATED APPLICATION

The present application is a 35 U.S.C. §371 national phase entry application of, and claims priority to, International Patent Application No. PCT/EP2012/001951, filed May 7, 2012, which claims priority to German Patent Application No. DE 102011103027.5, filed Jun. 1, 2011, the disclosures of which are hereby incorporated by reference in their entirety for all purposes.

### BACKGROUND

The invention relates to a new type of tooth replacement crown and tooth replacement bridge, which is comprised of several components, which are reversibly connected to each other by a macro-retentive bonding system, in which the components are preferably produced by means of computer-aided design and computer-aided manufacturing.

In particular, the invention relates to tooth replacement crowns and tooth replacement bridges, of which components are produced by means of computer-aided design and computer-aided manufacturing methods and in a subsequent mating step are put together by means of a reversible, macro-retentive connection geometry.

In dentistry, dental prostheses, such as bridges or full dentures are connected to implants and/or root caps by means of so-called “attachments”. Spherical male parts, on which female parts with retention elements are fitted or disengaged, amongst other things, are known for this purpose (see, for example: “Ball anchorage with friction-retentive grip from Schütz Dental GmbH, Rosbach). These anchoring elements serve to fix a removable dental prosthesis, which means that these connections are configured so that the dental prosthesis is recurrently detached and inserted.

The patent specification U.S. Pat. No. 7,114,952 pertains to an abutment for a dental implant with a prosthetic receiving tapered post with a locking step for removably restraining a sleeve member, which may be connected to a second sleeve member by means of a snap on mechanism. The patent specification does not describe the design and manufacture of reversibly fixed veneer structures on crown or bridge frameworks or implants.

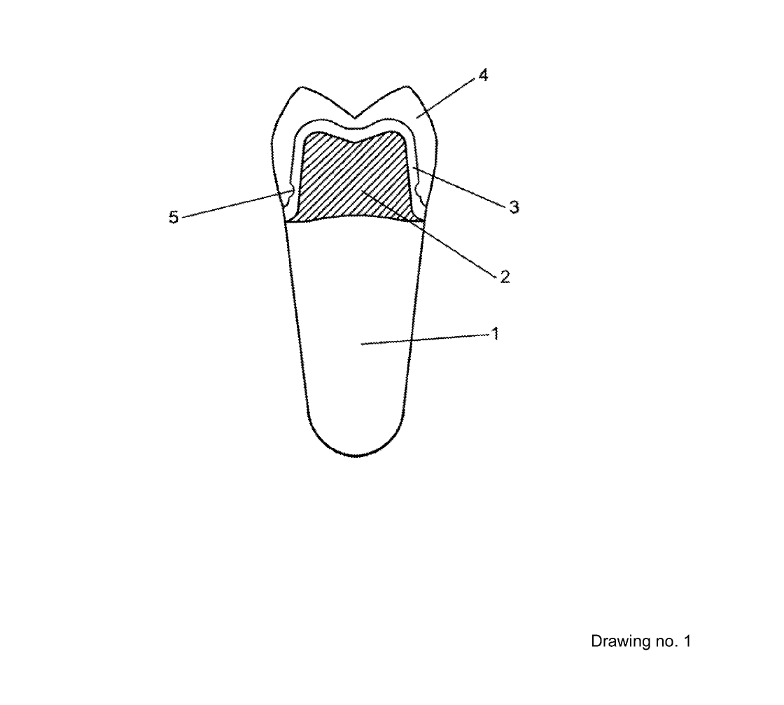

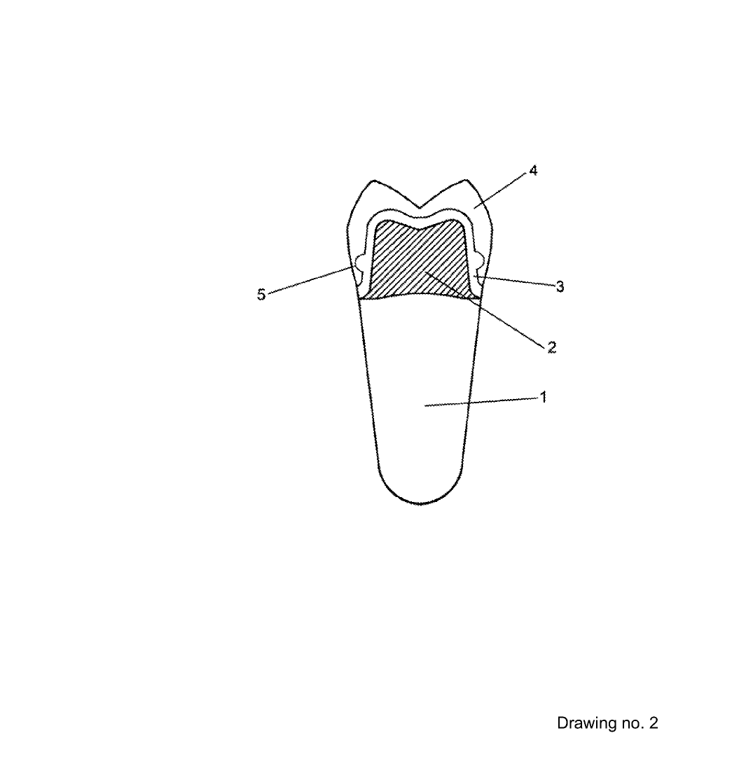

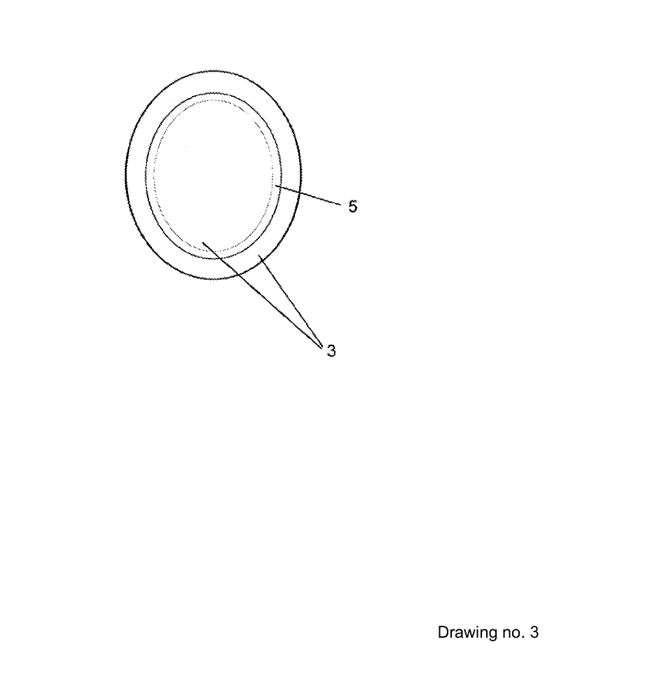

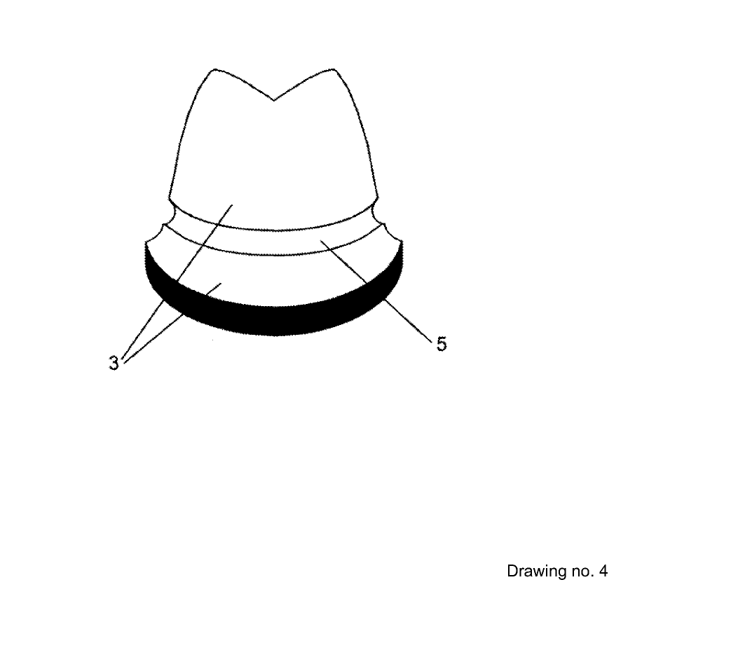

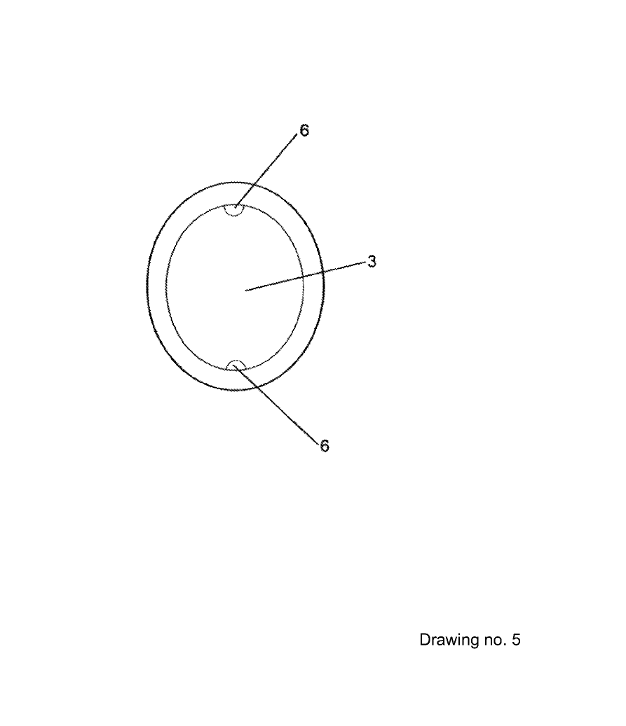

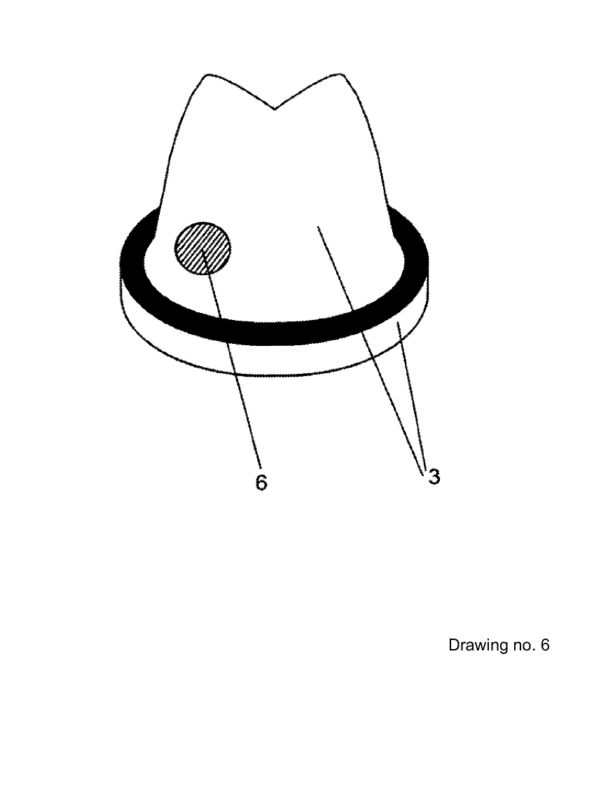

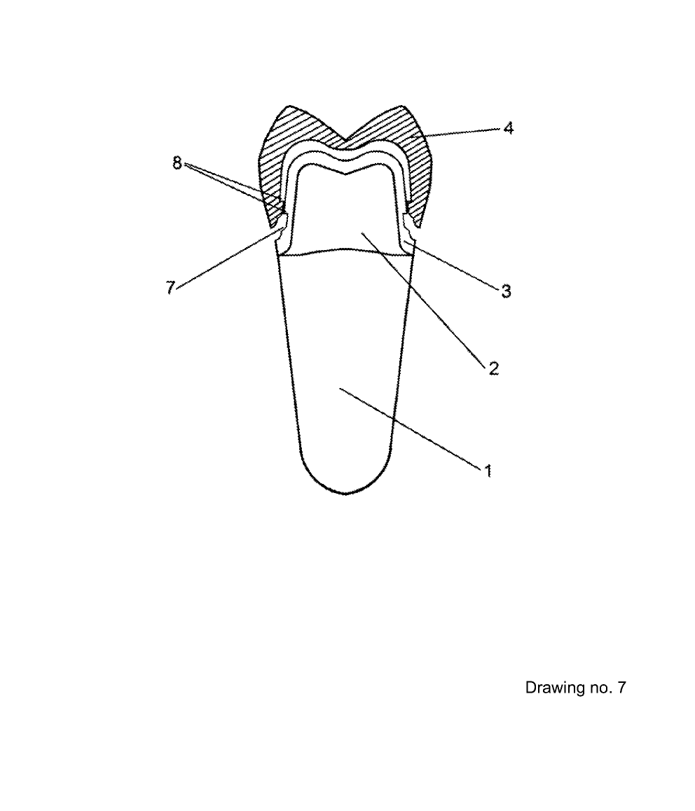

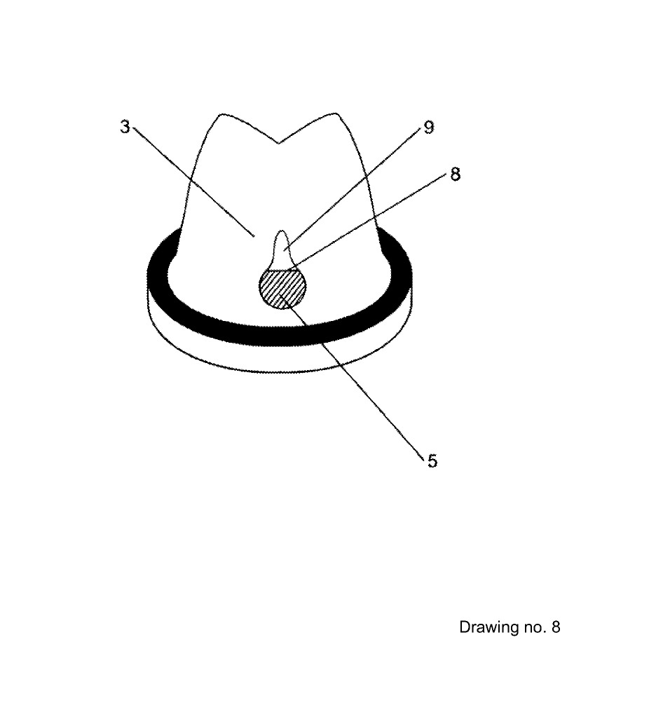

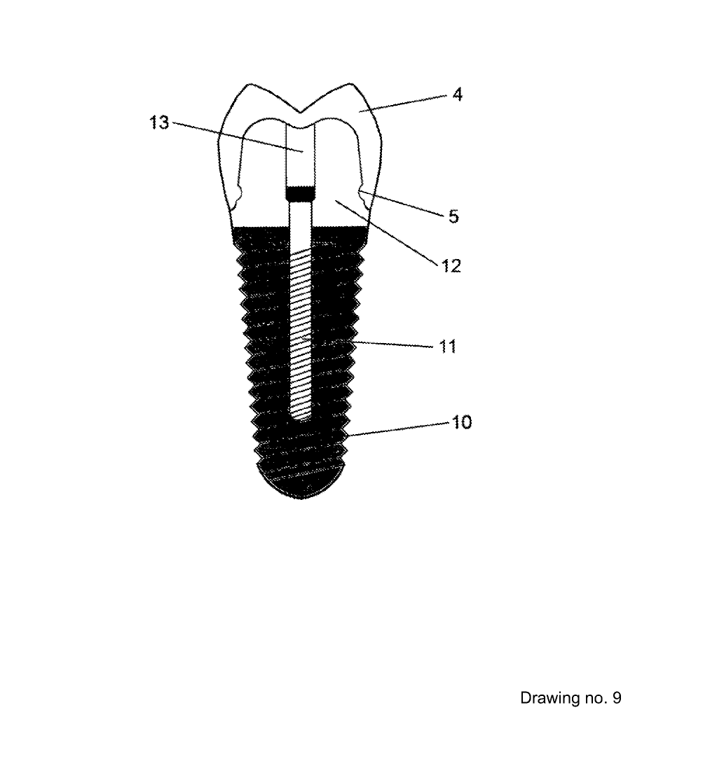

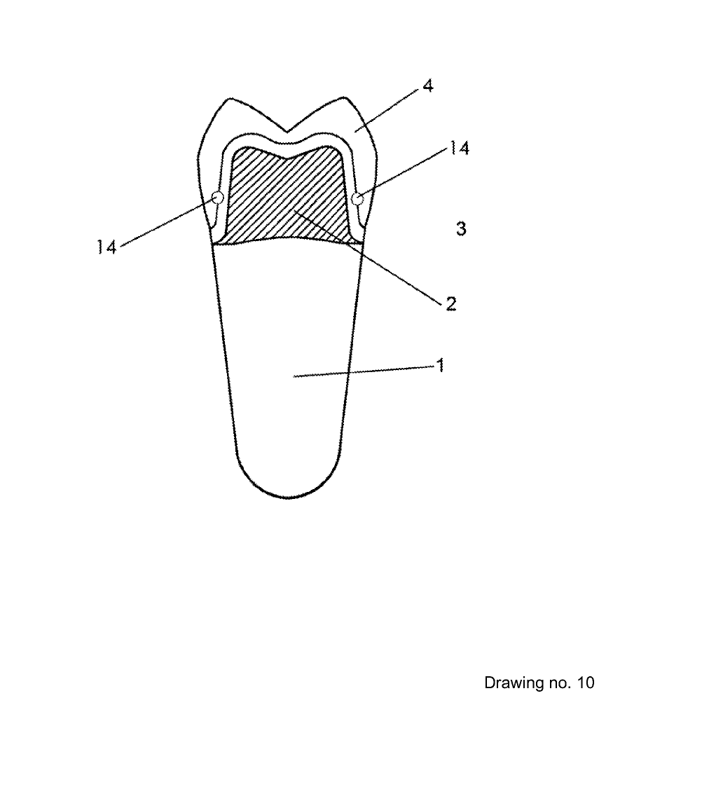

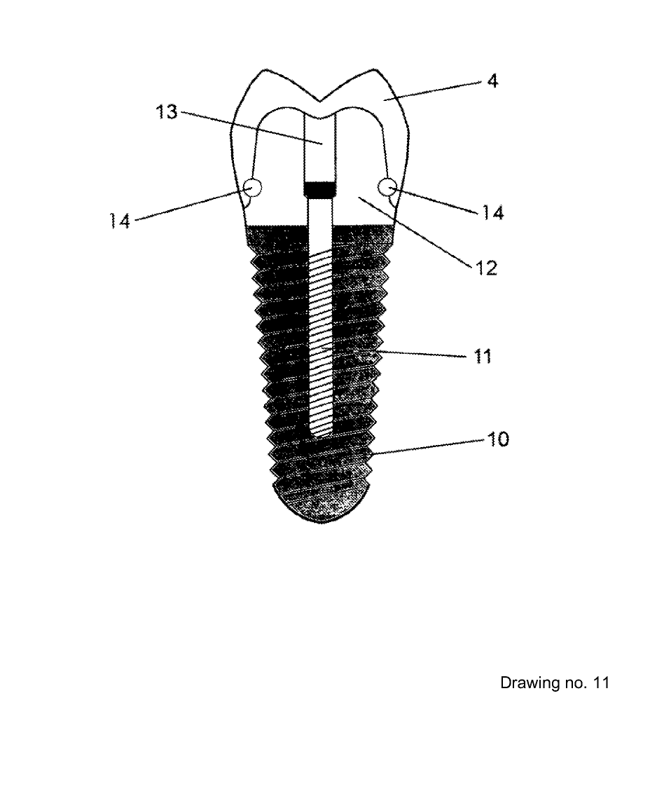

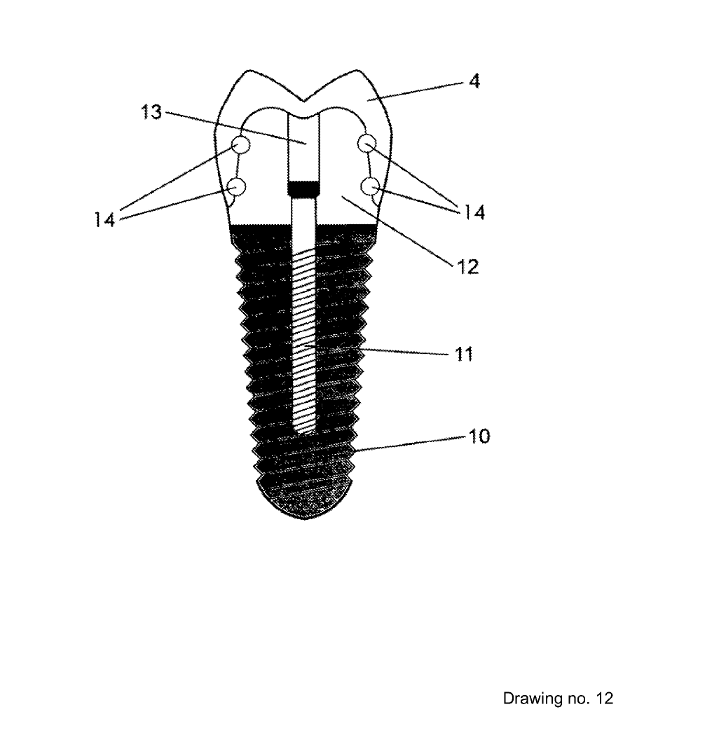

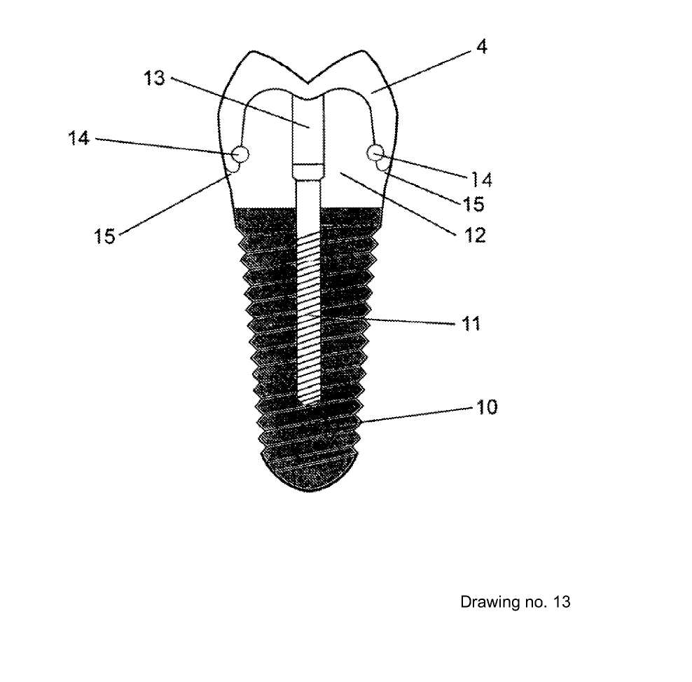

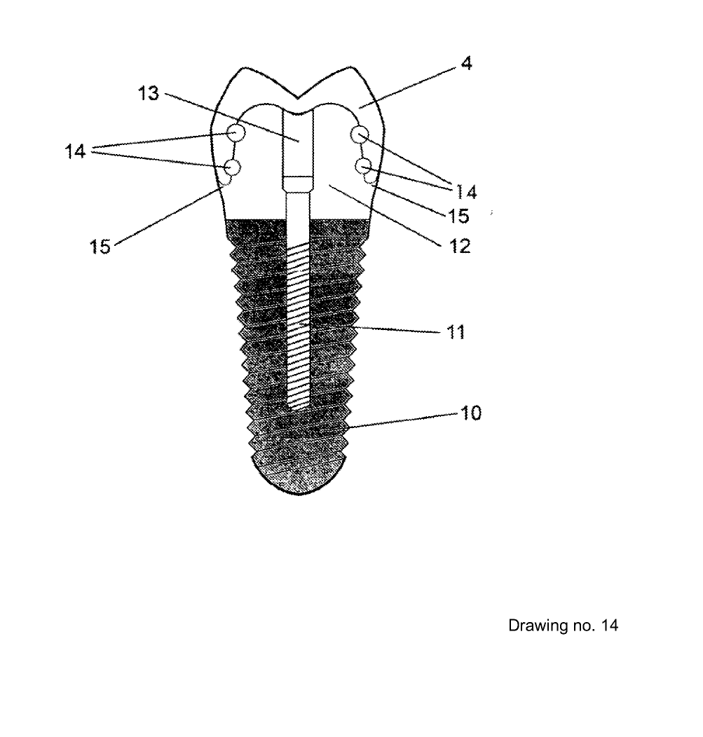

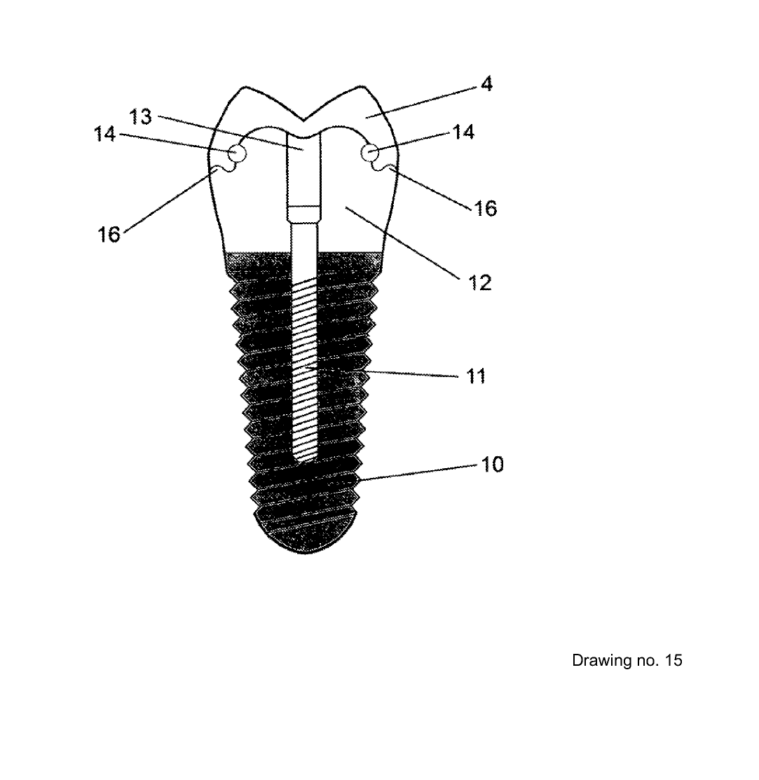

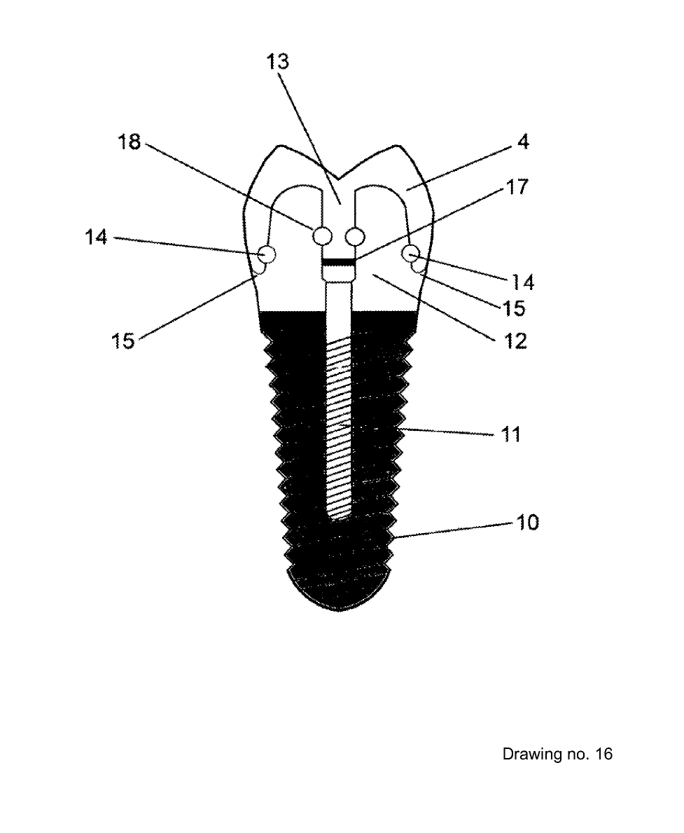

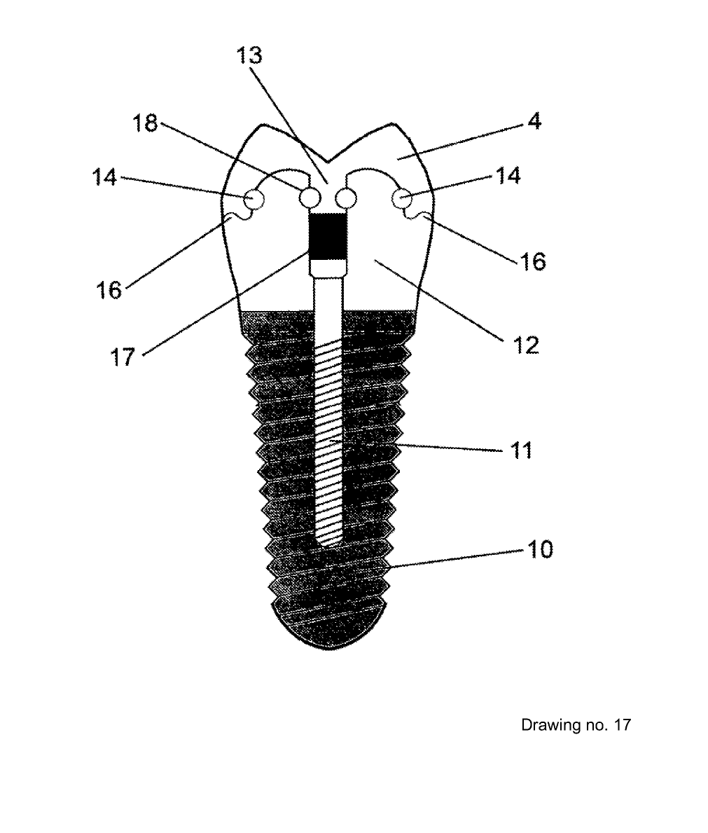

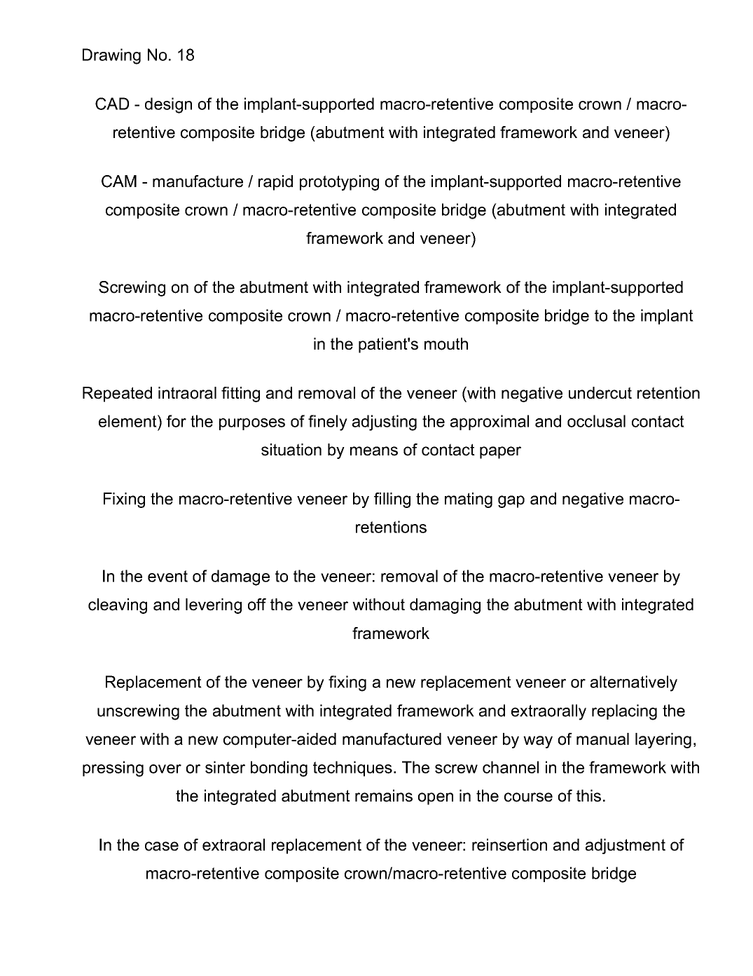

In patent application publication US 2010/0055645 FIGS. 13 and 14 show a dental implant together with a retention member and a cap. FIG. 24 discloses a retention member which is releasably engageable by means of a snap mechanism via retention means of the implant head and that releasably engages a cap via a snap mechanism. An anchoring system for fixing a dental prosthesis is described. The patent specification does not describe the design and manufacture of reversibly connected veneer structures on crown or bridge frameworks or implants.

A telescopic crown is known from DE 40 90 310, comprising a primary crown which may be fixed on a tooth stump and a secondary crown which may be slid thereon, in which a resilient retention element is retained in the inner wall of the secondary crown, which, with a latch projecting over the wall towards the outer wall of the primary crown, is inserted in an identically shaped latch recess in the outer wall of the primary crown in the desired position. According to claim 2 the latch and the latch recess are hemispherical in form. These described retention elements are not intended for fixed crowns or bridges, but rather are used as connection elements for removable dental prostheses in telescopic technology applications. Spring elements are used, which make it possible to detach and insert the removable dental prostheses on a constantly recurring basis. The retention element is a separate part which is integrated in the secondary structure of the telescopic crown. The patent specification does not describe the design and manufacture of reversibly fixed veneer structures on crown or bridge frameworks or implants.

The utility model DE 298 22 452 likewise discloses a generic device for fixing a dental prosthesis by means of a positive locking retention element working together with a fixed dental prosthesis or adapted residual teeth. These described retention elements are not intended for fixed crowns or bridges, but rather are used as connection elements for removable dental prostheses in telescopic technology applications. Spring elements are used, which make it possible to detach and insert the removable dental prostheses on a constantly recurring basis. The retention element is a separate part, which is mounted within the secondary structure of the telescopic crown. The patent specification does not describe the design and manufacture of reversibly fixed veneer structures on crown or bridge frameworks or implants.

In utility model DE 296 01 515 a double crown is disclosed, consisting of a primary crown which may be fixed to a tooth stump and a secondary crown which may be slid onto this, in which a resilient retention element is mounted in the inner wall of the secondary crown, which, with a latch projecting towards the outer wall of the primary crown may be inserted in a latch recess in the outer wall of the primary crown in the desired position. The utility model does not describe the design and manufacture of reversibly fixed veneer structures on crown or bridge frameworks or implants.

The utility model DE 201 03 480 shows a similar device. The described dental prosthesis is a telescopic crown with a resilient retention element. Telescopic crowns are retainers for removable dental prostheses. The utility model does not describe the design and manufacture of reversibly fixed veneer structures on crown or bridge frameworks or implants.

The utility model DE 20 2005 020 953 teaches a metallic dental restorative element, characterised in that it is produced according to a method, in which a control program is generated using the data of the previously prepared desired shape of the dental situation, on the basis of the calculated tool movements required for manufacturing purposes, which causes a numerically controlled manufacturing unit to generate a positive image of the desired shape of the dental situation from a burnable and non swelling material, in which the numerically controlled manufacturing unit comprises at least one production tool. According to claim 2 the manufacturing unit comprises a milling and grinding unit. According to claim 4 the manufacturing unit is directly computer controlled. According to claim 5 the control program is rendered in a CNC program. According to claim 7 the patient's dental situation is recorded by means of a scanner. According to claim 11 a virtual, three-dimensional image of the dental situation is generated using the acquired data. According to claims 12 and 13 the desired shape of the dental situation is produced by the operator or automatically by computer software or largely automatically on the basis of the data stored in a database. The patent specification does not describe the design and manufacture of reversibly fixed veneer structures on crown or bridge frameworks or implants.

A dental crown or a dental implant is known from DE 10 2008 009 793 A1 with a primary crown which may be connected in a friction locking and/or positive locking manner with a treated tooth or for the purposes of implantation with a basic support or support posts which may be implanted in a jaw and has a secondary crown which surrounds the primary crown. A mobility of the secondary crown in relation to the primary crown in a plane parallel to the occlusal plane is described. Elastic deformations of the jawbone are not impeded by this mobility of the dental prosthesis or dental bridge. The patent specification does not describe the design and manufacture of reversibly fixed veneer structures on crown or bridge frameworks or implants.

In the aforementioned applications either elements for removable dental prostheses (e.g. telescopic crowns or double crowns) or anchoring systems for removable dental prostheses on implants are described. None of the patent specifications mentioned above refer to the reversible fixing of veneer structures on crown or bridge frameworks or implants.

At present it is possible for multi-part composite crowns to be produced by means of computer-aided methods. In patent applications DE-10 2005 042 091 A1 and WO-0002007 028787 AI this kind of crown and bridge as well as the method for the production thereof is described. The production of the individual components, thus the framework and the veneer, is effected in this context by means of CAD design and CNC milling. Both parts are subsequently irreversibly joined, in which the joining may either be effected by way of ceramic sinter bonding firing or plastic adhesive bonding. In EP 1992302 A1 a two-part crown bridge design is known, with the parts likewise being irreversibly connected to each other by a sinter bonding step, in which here, as a distinctive feature, the veneer is milled from a pre-sintered silicate ceramic block and only densely sintered during the joining process.

What these composite crown appliance types have in common is that both individual components are irreversibly connected to each other, which means that the veneer may no longer be removed from the framework without destroying the complete restoration. The ability for the veneer to be attached and detached repeatedly would eliminate this disadvantage. In dental crown and bridge therapy the situation often occurs where veneers (ceramic, plastic) have damage due to masticatory loading, e.g. cracks or veneer chipping. Repairing these defects is only possible in the mouth, although the aesthetic outcomes and durability of these repairs is often to be considered as unsatisfactory.

A further problem consists in the fact that ceramic veneer materials often exhibit a considerably greater hardness than natural dental enamel, whilst veneer plastics exhibit a lesser hardness. In order to avoid the undesirably rapid wear of the veneer it is therefore preferable for ceramic veneers to be used. Owing to the brittleness of ceramic materials these often tend to crack and chip. This effect primarily manifests itself in implant-supported ceramic restorations. Veneer crowns and bridges on implants represent a special case, which are either screwed or cemented on so-called “abutments”. If these implant restorations are screwed to the implant there is at least the possibility of reversibly removing the crown or bridge from the mouth. However, a ceramic repair of the ceramic veneer material is mostly not possible, as with the high temperatures of ceramic firing the degassing of protein inclusions in the ceramic pores may occur, and thus leading to the destruction of the veneer. Thus, in most cases solely a repair using plastic may be carried out, which especially in a composite with ceramic may cause recurring problems. Should a restoration nevertheless be cemented onto an implant abutment, in this case there is only the possibility of an intraoral veneer repair with the problems as mentioned. In particular, implant-supported, ceramic-veneered restorations in the systems that have hitherto been on the market exhibit an increased tendency to fracture or chip in the ceramic veneer. This is predominantly due to the fact that implants have no protective mechanism via a reflex arc, thus there are no overload receptors, which are located in the periodontium in the case of a natural tooth, to accommodate non-physiological forces and therefore also protect the artificial dental crown against consequential damage. In the interim the possibility has presented itself of using high strength, fully anatomical zirconium dioxide crowns on implants, which are tooth-coloured, but do not have a ceramic veneer layer. Another possibility for avoiding damage to veneer are sintered composite crowns and bridges, which comprise a framework and veneer, with both units being manufactured using the CAD/CAM method and joined together by sinter bonding firing to form a highly stable restoration. Both the fully anatomical, zirconium dioxide restorations and the sintered composite crowns and bridges are so stable that damage to crowns and bridges due to extreme loads when in use on implants can for the most part be avoided. However, there is then a risk that severe damage may occur in the implant fixed within the bone. In the worst case scenario implants damaged in this way must be explanted.

To sum up, it can be said that ceramically veneered crowns and bridges on implants are especially subject to increased loading. If the stability of these appliances is increased the weakest member is shifted directly in the implant, so that damage to the same may occur as a result.

### SUMMARY

In the face of these disadvantages of the state of the art the task of the invention consists in creating a crown and/or bridge appliance which facilitates the removal of the veneer structure from the framework structure without damaging or destroying the framework.

This problem is solved according to the invention by the features of independent claims 1 and 29, in which expedient further developments are characterised by the respective dependent claims.

### BRIEF DESCRIPTION OF THE DRAWINGS

In Drawings no. 1 to no. 18 the application of the invention is explained, these show:

Drawing no. 1—a section through a natural lateral tooth with a macro-retentive composite crown in the view from the interdental perspective

Drawing no. 2—the same elements as Drawing no. 1, although here the retention element is configured with positive geometry in the crown framework

Drawing no. 3—occlusal top view of the crown framework

Drawing no. 4—the same framework shown in perspective

Drawing no. 5—also the occlusal top view of a crown framework

Drawing no. 6—the same framework shown in perspective

Drawing no. 7—interdental sectional view of a configuration of the retention elements

Drawing no. 8—buccal/palatinal view of the framework

Drawing no. 9—the configuration of a screwed macro-retentive composite crown on an implant

Drawing no. 10—section though a natural lateral tooth with a macro-retentive composite crown with an undercut retention element in both the crown framework and in the veneer in the view from the interdental perspective

Drawing no. 11—the configuration of a macro-retentive composite crown on an implant screwed to an undercut retention element in both the crown framework and in the veneer in the view from the interdental perspective

Drawing no. 12—the configuration of a macro-retentive composite crown on an implant screwed to two mutually superimposed undercut retention elements in both the crown framework and in the veneer in the view from the interdental perspective

Drawing no. 13—the configuration of a macro-retentive composite crown on an implant screwed to an undercut retention element in both the crown framework and in the veneer and retentive configuration of the transition area from the framework to the veneer in the view from the interdental perspective

Drawing no. 14—the configuration of a macro-retentive composite crown on an implant screwed to two mutually superimposed undercut retention elements in both the crown framework and in the veneer and retentive configuration of the transition area from the framework to the veneer in the view from the interdental perspective

Drawing no. 15—the configuration of a macro-retentive composite crown on an implant screwed to an occlusal veneer and with an undercut retention element in both the crown framework and in the veneer and special retentive configuration of the transition area from the framework to the veneer in the view from the interdental perspective

Drawing no. 16—the configuration of a macro-retentive composite crown on an implant screwed to an undercut retention element in both the crown framework and in the veneer and retentive configuration of the transition area from the framework to the veneer and an undercut retention element in the screw channel in both the crown framework and in the veneer in the view from the interdental perspective

Drawing no. 17—the configuration of a macro-retentive composite crown on an implant screwed to occlusal veneer and to an undercut retention element in both the crown framework and in the veneer and special retentive configuration of the transition area from the framework to the veneer and an undercut retention element in the screw channel in both the crown framework and in the veneer in the view from the interdental perspective

Drawing no. 18—the process sequence for producing a macro-retentive composite crown/macro-retentive composite bridge on implants and insertion in the patient as well as replacement of the veneer.

### DETAILED DESCRIPTION

The problems of the state of the art are solved by computer-aided methods being used to design and manufacture a crown or bridge, which comprises two parts, namely a framework and veneer, which may be detached from each other without damage to the framework. In this context the veneer serves as a wear and overload part and can be replaced when required. The connection is effected via macro-retentions, in the case of which material areas extending transversely to the crown axis create a connection, which is resistant to removal by being pulled off, between the framework and the veneer, as is the case for a “snap” mechanism, for example, so that the veneer is pressed on the framework during the fitting procedure against a briefly increased resistance, for example, in which the veneer sits in the final position without tension. For the purposes of sealing, either cement, composite material, plastics or silicones are used. Metals, plastics (particularly high performance polymers such as PEEK, for example), ceramics (glass ceramics, lithium disilicate, oxidic high performance ceramics, for example) can be used as the material for the framework, all tooth-coloured materials can be used as the material for the veneer, such as ceramics and plastics, for example, in which ideally plastics are used owing to the brittle fracture behaviour of ceramics. In a further configuration the macro-retentive element is attained by undercut areas which are formed in the veneer as well as in the framework, in which the macro-retentive effect is only achieved when both components the framework and veneer are connected by way of the undercut, macro-retentive areas being filled with plastics, composites or cements, for example. A connection of framework with macro-retentive areas and veneer with macro-retentive areas is also possible by using sinter bonding techniques by means of ceramic materials.

### Process Steps:

1. Data Acquisition

At the beginning of the treatment of the patient the existing oral situation is recorded. This can either be effected in analogue mode via conventional impression taking techniques or alternatively via digital casting by means of an intraoral 3D scanner. In conventional impressions dental plaster is poured out into the impression, so that an identical image of the jaw and teeth results. These plaster models are subsequently captured by means of a 3D laboratory scanner, so that a 3D data set of the oral situation results therefrom. Mechanical digitizing scanners as well as photo-optical laser and structured light scanners are deployed in the acquisition process. Micro CT scanners form a distinct category and with these it is also possible to directly scan in the dental impression, without this having plaster poured into it. In the case of intraoral 3D scanners this data set is generated equally directly without further process steps.

2. CAD Design of Macro-Retentive Composite Crowns/Macro-Retentive Composite Bridges

On the basis of the digitalised jaw model, the ideal moulding of a dental prosthesis (crown, bridge) is designed on the tooth/teeth to be treated with the aid of a CAD program. The digital design of the anatomical external shape can be effected taking into account the static and dynamic occlusion (articulation). Using this fully anatomical data set as a basis, the software now generates a two-part data set, consisting of the framework and appurtenant veneer. In this context, the framework must have a conical angle of α/2>=0 degrees, so that the two parts can be joined together at all. Though ideally the design has defined, undercut areas, which make it possible to “snap in” the veneer on the framework. The configuration of these “snap-in latches” forming the macro-retentive areas can take all manner of shapes, in which they are determined by the material and the geometry of the crown. This means that the higher the modulus of elasticity of the veneer material is, the smaller the depth of the macro-retentive areas should be. Likewise the vertical length of the veneer determines the depth of the latches. The positioning and geometric shaping of the macro-retentive areas can be effected by way of being defined by an operator in the design software or be automatically suggested by the design program and then confirmed by the operator.

The “snap-in latches” can either be shaped circular throughout in the circumferential direction of the crown or bridge framework or alternatively they are only present in defined sectors of the frameworks. In the longitudinal section of the crown there are likewise all manner of possibilities for configuring the “snap-in latches”. Thus, it is possible, for example, to configure the latch in the shape of a circular segment, as seen in this longitudinal section. Likewise it is possible only to create local retention points, which have the shape of segments of a hemisphere, in which these retention points have feed grooves for improved fine adjustment, which however generate increasing resistance within the feed path so as to finally end in the snap-in spherical depression without tension. The exact configurations may be gathered from Drawings no. 1 to no. 9.

The external geometry of the framework defines the internal geometry of the veneer, in which for the case that a fixing material is to be used a mating gap can be provided for this fixing material.

A further configuration consists in both the framework structure on the framework exterior and the veneer on the veneer interior having a negative, undercut recess. This can, seen in the longitudinal section of the crown, be configured in various geometric shapes, particularly semicircular, semi-oval, semi-elliptic, semi-parabolic, square, rectangular, etc. The macro-retentive areas in the framework and veneer can be configured opposite or offset in relation to each other. The negative undercut macro-retentive areas can be formed in both the framework and in the veneer, in a further configuration the negative undercut macro-retentive areas can be formed either in the framework or in the veneer.

In the case of these configuration forms the veneer can be slid onto the framework and removed again. This offers the advantage that in the context of corrective measures during try-in on a model or patient the veneer may be removed by the dental technician or dentist. This is particularly important when adjusting appliances in the case of implants, as here the superstructure is screwed on the implant.

When an exact fit, particularly of the approximal and occlusal contact situation has been attained, in the course of definitive insertion the space between the veneer and framework (undercut recesses) is closed up by means of a filler material and the macro-retention arises. The filler material can be a plastic material or silicone material, for example. In particular, self curing plastics (self cure resins, PMMA materials), light curing composite material, dual curing composites, cements, etc. are suitable here. Silicone materials may also be used as a filler substance for the space. In the case of there being opposing recesses and a circular configuration, the space can still be filled by means of a prefabricated ring, in which this ring is pressed onto the framework until it snaps into the undercut recess and subsequently the veneer is fitted until its undercut recess also snaps into the prefabricated ring placed on the framework.

The connecting material also has the task of sealing the marginal gap between the veneer and framework. In addition, active agents may be added to the connecting material, such as substances which counteract the build-up of dental place or substances which bring about a remineralisation and strengthening of the hard tooth tissue, for example.

3. Producing Macro-Retentive Composite Crowns/Macro-Retentive Composite Bridges by Means of a Subtractive Method (e.g. CNC Milling) or by Means of an Additive Method (e.g. Rapid Prototyping, Such as 3D Printing, Laser Sintering, Stereolithography, Etc.).

The production of macro-retentive composite crowns/macro-retentive composite bridges is effected on the basis of the data designed in CAD either by means of CNC milling machines and/or by additive rapid prototyping methods, such as laser sintering, stereolithography or 3D printing, for example. Combining the production of a crown or bridge geometry by means of an additive rapid prototyping method or subtractive milling method in a burn-out material with a subsequent casting or compression moulding technique in a “lost wax” procedure is possible.

A considerable advantage of the invention consists in the fact that for the first time in the case of veneered crowns or bridges if damage to the veneer occurs it is easy for it to be removed and replaced with an identically configured replacement veneer. The data for this are generated digitally by the CAD design process and saved. Likewise, by means of the invention it is possible to incorporate initially defined predetermined breaking point values in the form of the macro-retentive veneer in implant appliances, so that in the event of overloading solely the veneer is destroyed, whilst the framework, the implant abutment and above all the implant as well as the osseous bed remain undamaged. A defined predetermined breaking point value can be attained by way of a specific thickness/gauge of the veneer, or else by a geometric predetermined breaking point. The size and the position of the geometric predetermined breaking point or the thickness of the veneer and the maximum load prior to breaking, which is dependent thereon, should ideally be calculated by suitable software. Software which can carry out FEM calculations, for example, would be suitable for this purpose. A further advantage comes to the fore with respect to screwed veneer crowns and bridges on implants. In this instance, it is possible for the first time to design the implant abutment already as a framework with snap-in latches. The abutment can be designed to be one-part or two-part with a base, with which the abutment can, for example, be adhesive bonded or sintered by means of fusion ceramics or solder glass. Metals, plastics or ceramics (glass ceramics, lithium disilicate, oxide ceramics, such as zirconium dioxide or aluminium oxide, for example) are conceivable as the material for the abutment. Ideally the abutment/framework should be tooth-coloured. After the abutment has been screwed onto the implant, the veneer can be “clicked onto” the abutment, in which it is expedient to use composite material or cement for the purposes of sealing. An infiltration of germs from the oral environment via the screw channel, which has indeed been possible until now in the case of screwed crowns and bridges, can be completely prevented by this procedure, so that the prognosis for the implants may be improved. Designing undercut macro-retentions in a framework and veneer with subsequent filling upon insertion of the restoration offers the advantage that prior to insertion the veneer may be detached and corrective measures may be undertaken. This advantage is particularly important in the case of screwed superstructures on implants, as otherwise in this case there is no possibility of undertaking any corrections of the approximal contacts. Moreover, the finishing and polishing of chewing surfaces outside the mouth is easier and more efficient. Using ceramic veneer structures means ceramic firing, such as glaze firing, for example, may also be carried out once again at any time. The connection of the framework with macro-retentive areas and the veneer with macro-retentive areas is also possible by way of sinter bonding techniques using ceramic materials.

In the case of implant-supported dental prostheses the possibility likewise exists of a veneer made of plastic being removed from the framework after a trial period of wear, the screwed framework being unscrewed from the implant and subsequently a ceramic veneer being applied to the framework by means of manual layering, press over techniques or sinter bonding firing. Because of this, in the case of implants a trial phase with screwed crowns and bridges with plastic veneers can be carried out initially, with these then being replaced with ceramic veneers after the trial phase.

The following should be noted as considerable advantages compared with the state of the art:
1. The possibility of replacing a damaged or defective veneer with an identical CAD/CAM veneer without difficulty
2. Integration of an overload element in the crown and bridge prosthesis, so that the natural abutment teeth or implants as well as the crown and bridge frameworks are protected.
3. New, screwed implant crowns/implant bridges, which have improved sealing in the area of the screw connection. Moreover, the actual framework structure is omitted here.

Drawing no. 1 shows a longitudinal section through a lateral tooth with a macro-retentive composite crown in the view from the interdental perspective, with the tooth root (1), the prepared tooth stump (2), framework (3) and the appurtenant veneer (4). The retentive element (5) is depicted here by way of an example in this longitudinal section as a circular segment. Other geometric configurations of the retention element are possible, the height position of this element in the framework can also be varied, as can the actual size (dimensions in X, Y and Z direction, with Z parallel to the crown axis), thus e.g. the diameter and depth of the retention element. In Drawing no. 1 the retention element has a negative geometric configuration in the crown framework.

In principle, drawing no. 2 shows the same elements as drawing no. 1, although here the retention element has a positive geometric configuration in the crown framework.

Drawing no. 3 shows an occlusal top view of the crown framework, with the components—the framework (3) and circular retentive element (5)

Drawing no. 4 shows the same framework shown in perspective with framework (3) and circular retentive element (5)

Drawing no. 5 likewise shows the occlusal top view of a crown framework, although with an arc-shaped configuration of the retention element (6) limited to the buccal and palatinal area.

Drawing no. 6 shows the same framework shown in perspective with the framework (3) and the arc-shaped retention element (6).

Drawing no. 7 shows the interdental sectional view of a configuration of the retention element with a special locking edge (8). In the drawing the tooth root (1), the prepared tooth stump (2), the framework (3) and the appurtenant veneer (4), the specialised retentive element (7) with locking edge (8) can be seen.

Drawing no. 8 shows the buccal/palatinal view of the framework (3), with retention spherical depression (5), locking edge (8) and insertion path (9).

Drawing no. 9 shows the configuration of a macro-retentive composite crown on an implant screwed in the view from the interdental perspective in sectional view, with the components implant (10), implant screw (11), framework/abutment (12), retention element (5) and veneer (4) as well as screw channel (13).

Drawing no. 10 shows the longitudinal section parallel to the Z direction through a lateral tooth with macro-retentive composite crown in the view from the interdental perspective, with the tooth root (1), the prepared tooth stump (2), the framework (3) and the appurtenant veneer (4). The retentive element (14) has an undercut negatively geometric configuration in both the crown framework and in the veneer.

The retentive element undercut on both sides (14) is depicted here as being circular by way of an example in this cross section. Other geometric configurations of the retention element are possible, the height position of this element in the framework can also be varied, as can the actual size (dimensions in X, Y and Z direction), thus e.g. the diameter and depth of the retention element as well as the number of retention elements.

Drawing no. 11 shows the configuration of a macro-retentive composite crown on an implant screwed in the view from the interdental perspective in the longitudinal section parallel to the Z direction, with the components implant (10), implant screw (11), framework/abutment (12), veneer (4) and screw channel (13) as well as the undercut retentive element (14) with negative geometry in both the crown framework and in the veneer.

Drawing no. 12 shows the configuration of a macro-retentive composite crown on an implant screwed in the view from the interdental perspective in the longitudinal section parallel to the Z direction, with the components implant (10), implant screw (11), framework/abutment (12), veneer (4) and screw channel (13) as well as mutually superimposed undercut retentive elements (14) with negative geometry in both the crown framework and in the veneer.

Drawing no. 13 shows the configuration of a macro-retentive composite crown on an implant screwed in the view from the interdental perspective in the longitudinal section parallel to the Z direction, with the components implant (10), implant screw (11), framework/abutment (12), veneer (4), screw channel (13) as well as the undercut retentive element (14) with negative geometry in both the crown framework and in the veneer and the retentive configuration of the transition area from the framework to the veneer (15).

Drawing no. 14 shows the configuration of a macro-retentive composite crown on an implant screwed in the view from the interdental perspective in the longitudinal section parallel to the Z direction, with the components implant (10), implant screw (11), framework/abutment (12), veneer (4), screw channel (13) as well as mutually superimposed undercut retentive elements (14) with negative geometry in both the crown framework and in the veneer and the retentive configuration of the transition area from the framework to the veneer (15).

Drawing no. 15 shows the configuration of a macro-retentive composite crown with an occlusal veneer on an implant screwed in the view from the interdental perspective in the longitudinal section parallel to the Z direction, with the components implant (10), implant screw (11), framework/abutment (12), veneer (4), screw channel (13) as well as the undercut retentive element (14) with negative geometry in both the crown framework and in the veneer and the special retentive configuration of the transition area from the framework/abutment to the occlusal veneer (16).

Drawing no. 16 shows the configuration of a macro-retentive composite crown on an implant screwed in the view from the interdental perspective in the longitudinal section parallel to the Z direction, with the components implant (10), implant screw (11), framework/abutment (12), veneer (4), screw channel (13), the undercut retentive element (14) with negative geometry in both the crown framework and in the veneer, the retentive configuration of the transition area from the framework to the veneer (15), the sealing washer on the screw head (17) as well as the undercut retentive element in the screw channel (18) with negative geometry in both the crown framework and in the veneer.

Drawing no. 17 shows the configuration of a macro-retentive composite crown with occlusal veneer on an implant screwed in the view from the interdental perspective in the longitudinal section parallel to the Z direction, with the components implant (10), implant screw (11), framework/abutment (12), veneer (4), screw channel (13), the undercut retentive element (14) with negative geometry in both the crown framework and in the veneer, the special retentive configuration of the transition area from the framework/abutment to the occlusal veneer (16), the sealing washer on the screw head (17) as well as the undercut retentive element in the screw channel (18) with negative geometry in both the crown framework and the occlusal veneer.

Drawing no. 18 shows the process sequence for producing a macro-retentive composite crown/macro-retentive composite bridge on implants and insertion in the patient as well as the replacement of the veneer in the case of a defect in the veneer or in the case of the replacement of the veneer with another material, particularly ceramic materials.

1. A dental prosthesis, including individual components of a framework and veneer, characterised in that the veneer is configured to be removed from the framework without damaging the framework.

2. The dental prosthesis according to claim 1, characterised in that the design and manufacture of the framework and veneer is effected by means of computer-aided methods.

3. The dental prosthesis according to claim 1, characterised in that tooth-coloured materials are used as the material for the veneer, including glass ceramics, high performance oxidic ceramics and plastic.

4. The dental prosthesis according to claim 1, characterised in that the material groups metals, ceramics and plastics are used as the material for the framework.

5. The dental prosthesis according to claim 1, characterised in that cement, composite material, plastics or silicones are used for the purposes of sealing between the framework and veneer.

6. The dental prosthesis according to claim 1, characterised in that the veneer serves as a wear and overload part and is configured to be replaceable.

7. The dental prosthesis according to claim 1, characterised in that the dental prosthesis has a defined predetermined breaking point which is defined by the geometric configuration of the veneer or the thickness of the veneer.

8. The dental prosthesis according to claim 1, characterised in that on the basis of the predetermined breaking point value the geometric configuration of the veneer is calculated by a computer program.

9. The dental prosthesis according to claim 1, characterised in that a three-dimensional external geometry of the framework is congruent in shape to a three-dimensional internal geometry of the veneer.

10. The dental prosthesis according to claim 1, characterised in that provision has been made for a mating gap for a fixing material between the framework and veneer.

11. The dental prosthesis according to claim 1, characterised in that a proesthesis design includes undercut areas, enabling the veneer to snap in on the framework.

12. The dental prosthesis according to claim 1, characterised in that a configuration of retention elements is effected in one or more geometric shapes.

13. The dental prosthesis according to claim 12, characterised in that a three-dimensional calculation and design of the retention elements is effected taking into account material properties of the framework and veneer and having regard to an overall geometry of a crown.

14. The dental prosthesis according to claim 12, characterised in that a retention element of the retention elements has a circular configuration around the crown or a bridge framework.

15. The dental prosthesis according to claim 12, characterised in that a retention element of the retention elements is limited to defined sectors of the framework and the veneer.

16. The dental prosthesis according to claim 12, characterised in that a retention element of the retention elements going from the chewing surface, seen in cross-section, is configured in a shape of a circular segment.

17. The dental prosthesis according to claim 12, characterised in that the retention elements are punctiform and circular, in which these retention points have feed grooves for improved fine adjustment of the veneer.

18. The dental prosthesis according to claim 12, characterised in that the retention elements have a locking edge with an occlusal orientation.

19. The dental prosthesis according to claim 1, characterised in that a veneer structure is configured as an occlusal chewing surface.

20. The dental prosthesis according to claim 1, characterised in that retentive elements are configured as undercut negative geometries in the framework structure or in a veneer structure.

21. The dental prosthesis according to claim 1, characterised in that the veneer structure provided with undercut retentive elements are detachable from the framework structure provided with undercut retentive elements without damage, including during the try-in phase in the model and patient.

22. The dental prosthesis according to claim 1, characterised in that undercut retentive elements in the framework structure or in a veneer structure are filled with light curing or self curing materials.

23. The dental prosthesis according to claim 22, characterised in that plastics, cements or silicones are used as filler materials for the undercut retentive elements.

24. The dental prosthesis according to claim 1, characterised in that the veneer is configured to serve as a predetermined breaking point or overload protection for dental prosthetic appliances, including implants.

25. The dental prosthesis according to claim 1, including an implant abutment configured as a crown framework with snap-in latches for a veneer structure.

26. The dental prosthesis according to claim 1, characterised in that through sealing of a screw channel in case of implant abutments by means of a sealing washer or composite material or cement an infiltration of germs from an oral environment via the screw channel into the implant is prevented.

27. The dental prosthesis according to claim 1, characterised in that in case of screwed implant crowns and implant bridges retentive elements are formed in the screw channel in the framework structure or in a veneer structure as undercut retentive geometries.

28. The dental prosthesis according to claim 1, characterised in that a replacement of the veneer, including a damaged or defective veneer, is effected with an identical veneer, produced using a computer-aided method.

29. A method for producing a dental prosthesis, comprising;
producing-individual components of a framework and reversibly removable veneer by means of computer-aided design methods and by means of computer-aided manufacturing methods.

30. The method for producing a dental prosthesis according to claim 29 further comprising:
effecting a three-dimensional calculation and design of a geometry of the retention elements taking into account the material properties of the framework and veneer and having regard to an overall geometry of a crown.

31. The method for producing a dental prosthesis according to claim 29, further comprising effecting a replacement of a veneer, including a damaged or defective veneer with an identical veneer, produced using a computer-aided method.

32. The method for producing a dental prosthesis according to claim 29, further comprising: effecting a replacement of the veneer, including a replacement of the veneer in-dental prostheses screwed on implants, after the unscrewing of the dental prosthesis from the implant, by way of manual layering of plastic materials or ceramic veneer materials, by way of the press over technique with ceramic materials or plastic materials or by way of sintering on a veneer produced using a computer aided method by means of sinter bonding techniques.
